(19) United States
(12) Patent Application Publication
(10) Pub. No.: US 2025/0342908 A1
(43) Pub. Date: Nov. 6, 2025

(54) TECHNOLOGIES FOR PREDICTING PHENOTYPE AND ASSOCIATED BIOLOGICAL PATHWAYS FROM GENOMIC VARIATION DATA

(71) Applicant: BATTELLE MEMORIAL INSTITUTE, Columbus, OH (US)

(72) Inventors: Emily Anible, Columbus, OH (US); Chris Dibble, Columbus, OH (US); Steven Higgins, Columbus, OH (US); Robert Murdoch, Columbus, OH (US); Mughilan Muthupari, Boyds, MD (US)

(21) Appl. No.: 19/197,418

(22) Filed: May 2, 2025

Related U.S. Application Data
(60) Provisional application No. 63/642,014, filed on May 3, 2024.

(51) Int. Cl. G16B 20/20; G06N 20/20; G16B 45/00; G16B 50/30; G16H 40/20

(52) U.S. Cl. CPC G16B 20/20; G06N 20/20; G16B 45/00; G16B 50/30; G16H 40/20

(57) ABSTRACT

Technologies for predicting phenotype and associated biological pathways from genomic variation data are disclosed. According to one aspect of the disclosure, a method may include converting, by a compute device, data indicative of genomic variation into images. The method may also include applying, by the compute device, a machine learning model to the images to identify relationships between the genomic variation and phenotypic variation. Further, the method may include determining, by the compute device, one or more impactful genomic regions that underlie an identified relationship between a genotype and a phenotype.

### CROSS-REFERENCE TO RELATED APPLICATION

This application claims the benefit of U.S. Provisional Patent Application No. 63/642,014, filed May 3, 2024, the entire disclosure of which is incorporated herein by reference.

### STATEMENT REGARDING FEDERALLY SPONSORED RESEARCH

This invention was made with government support under Grant No. HR0011-23-9-0055 from the Defense Advanced Research Projects Agency. The government has certain rights in the invention.

### BACKGROUND

Genome-wide association studies (GWAS) are increasingly the toolkit of choice for identifying candidate genetic drivers of phenotype. However, GWAS have several downsides. First, GWAS are data-intensive. They require large amounts of data about both traits in a desired study population, as well as the collection of high-quality genomic data. This can be cost- and time-prohibitive, particularly in non-model organisms. GWAS, for instance, are generally performed on high-performance computing clusters (HPC), rather than on standard workstations. This limits their availability to those with HPC access. Second, GWAS is often unable to identify causal variants and genes. That is, many significant GWAS associations, once adjusted for multiple comparison, may arise for a single trait of interest. These numerous “hits” may involve genes or regions of little individual significance, thereby making it difficult to determine which, if any, cause deviation in the trait.

### SUMMARY

According to one aspect of the disclosure, a compute device includes circuitry configured to convert data indicative of genomic variation into images. The circuitry may also be configured to apply a machine learning model to the images to identify relationships between the genomic variation and phenotypic variation. In addition, the circuitry may be configured to determine one or more impactful genomic regions that underlie an identified relationship between a genotype and a phenotype. In some embodiments, the circuitry may be configured such that converting data indicative of genomic variation into images includes converting data indicative of genomic variation into greyscale images. The circuitry may also be configured such that converting data indicative of genomic variation into images comprises to translate genome sequence data into k-mers. In some embodiments, the circuitry of the compute device is configured to convert data indicative of genomic variation into images by producing greyscale images indicative of position-indexed k-mers.

In some embodiments, the circuitry is configured such that converting data indicative of genomic variation into images includes generating a defined number of k-mer spectral images for each of multiple strains of an organism. Generating a defined number of k-mer spectral images for each of multiple strains of an organism may include generating a defined number of k-mer spectral images for each of multiple strains of a plant. In some embodiments, the circuitry may be configured such that generating a defined number of k-mer spectral images for each of multiple strains of a plant includes generating a defined number of k-mer spectral images for each of multiple strains of a crop. Further, in some embodiments, the circuitry may be configured such that generating a defined number of k-mer spectral images for each of multiple strains of a crop includes generating a defined number of k-mer spectral images for each of multiple strains of corn or rice. Converting data indicative of genomic variation into images may include utilizing long or short read sequence data (e.g. FASTQ-formatted). In some embodiments, the circuitry may be configured such that converting data indicative of genomic variation into images includes utilizing one or more variant call format files indicative of variations in a genome from a reference genome. In some embodiments, the circuitry may be configured such that converting data indicative of genomic variation into images includes utilizing raw genome sequence (FASTA-formatted) data.

In some embodiments, the circuitry may be configured such that converting data indicative of genomic variation into images includes utilizing k-mer counts of varying size k (e.g., 3, 5, and 7). The circuitry of the compute device may be configured to split an input file of nucleotide sequences into windows. The circuitry may be further configured to decompose the windows into sub-windows. Further, the circuitry may be configured to concatenate, within each sub-window, reference alleles and variant alleles and calculate k-mers within each sub-window. In some embodiments, the circuitry may be configured to compute pairwise Pearson correlation scores among the sub-windows to generate a correlation matrix for each window representing correlations between a variant and a reference sequence. The circuitry may be further configured to store the correlation matrix as an image for input to the machine learning model.

The circuitry may be configured such that computing pairwise Pearson correlation scores includes performing pairwise correlations between vectors of k-mer counts to generate a correlation matrix. The length of the vectors and matrix size may be determined by the k-mer size selected and the number of windows and sub-windows specified. For example, in some embodiments, the vectors may be of length 100 and the correlation matrix may be 100 by 100. In some embodiments, the circuitry may be configured such that storing the correlation matrix as an image includes storing the correlation matrix as a greyscale image for input to a neural network. In some embodiments, the circuitry may be configured such that applying a machine learning model involves applying an image recognition neural network to the images. The machine learning model may be a neural network and the circuitry may be further configured to train at least a portion of the neural network based on known genotype to phenotype relationships. In some embodiments, the circuitry may be configured such that applying a machine learning model includes providing, to the machine learning model, each of multiple k-mer spectral images for a genotype as corresponding channels of a multi-channel input image. In some embodiments, the machine learning model is a neural network and the circuitry is configured such that applying a machine learning model involves modifying the final layer of the neural network to output three values corresponding to probabilities of assignment to a high, medium, and low phenotypic trait value category.

In some embodiments, the circuitry is configured such that applying a machine learning model includes providing the images to an ensemble of neural networks. The circuitry, in some embodiments, may be configured such that providing the images to an ensemble of neural networks includes providing a subset of the images for a genotype to a first neural network and providing another subset of the images for the genotype to a second neural network. The circuitry, in some embodiments, may be configured to provide 3 k-mer images for the genotype to the first neural network and provide a remainder of the k-mer images for the genotype to the second neural network. In some embodiments, the circuitry may be configured such that determining one or more impactful genomic regions includes generating attribution scores associated with genomic regions. The attribution scores may indicate a degree to which the corresponding region contributed to or detracted from the identification of a relationship between the genotype and the corresponding phenotype.

In some embodiments, the circuitry may be configured such that determining one or more impactful genomic regions may include utilizing an integrated gradient algorithm to determine attribution scores. The circuitry may be configured such that determining one or more impactful genomic regions includes generating attribution images indicative of determined attribution scores. In some embodiments, the circuitry is further configured to identify clusters of pixels with values that satisfy a predefined threshold as the impactful regions that underlie the relationship between a genotype and a phenotype. The circuitry may, in some embodiments, be further configured to conduct pathway enrichment analysis based on the impactful genomic regions. The pathway enrichment analysis may include determining whether the impactful genomic regions are statistically associated with known biological pathways. In some embodiments, the circuitry may be configured to determine whether the impactful genomic regions represent novel pathways.

According to another aspect of the disclosure, a method includes converting, by a compute device, data indicative of genomic variation into images. The method may also include applying, by the compute device, a machine learning model to the images to identify relationships between the genomic variation and phenotypic variation. Further, the method may include determining, by the compute device, one or more impactful genomic regions that underlie an identified relationship between a genotype and a phenotype. In some embodiments, converting data indicative of genomic variation into images includes converting data indicative of genomic variation into greyscale images. Converting data indicative of genomic variation into images may include translating genome sequence data into k-mers. In some embodiments, converting data indicative of genomic variation into images includes producing greyscale images indicative of position-indexed k-mers. Converting data indicative of genomic variation into images may, in some embodiments, include generating a defined number of k-mer spectral images for each of multiple strains of an organism. Generating a defined number of k-mer spectral images for each of multiple strains of an organism may include generating a defined number of k-mer spectral images for each of multiple strains of a plant.

In some embodiments, generating a defined number of k-mer spectral images for each of multiple strains of a plant includes generating a defined number of k-mer spectral images for each of multiple strains of a crop. Generating a defined number of k-mer spectral images for each of multiple strains of a crop may, in some embodiments, include generating a defined number of k-mer spectral images for each of multiple strains of corn or rice. Converting data indicative of genomic variation into images may include utilizing long or short read sequence data (e.g. FASTQ-formatted). In some embodiments, converting data indicative of genomic variation into images includes utilizing one or more variant call format files indicative of variations in a genome from a reference genome. In some embodiments, converting data indicative of genomic variation into images includes utilizing raw genome sequence (FASTA-formatted) data. Converting data indicative of genomic variation into images, in some embodiments of the method, includes utilizing k-mer counts of varying size k (e.g., 3, 5, and 7). The method may also include splitting, by the compute device, an input file of nucleotide sequences into windows. Additionally, the method may include decomposing, by the compute device, the windows into sub-windows. Further, the method may include concatenating, by the compute device and within each sub-window, reference alleles and variant alleles. In addition, the method may include calculating, by the compute device, k-mers within each sub-window.

In some embodiments, the method includes computing, by the compute device, pairwise Pearson correlation scores among the sub-windows to generate a correlation matrix for each window representing correlations between a variant and a reference sequence. The method may also include storing, by the compute device, the correlation matrix as an image for input to the machine learning model. Computing pairwise Pearson correlation scores may include performing pairwise correlations between vectors of k-mer counts to generate a correlation matrix. The length of the vectors and matrix size may be determined by the k-mer size selected and the number of windows and sub-windows specified. For example, in some embodiments, the vectors may be of length 100 and the correlation matrix may be 100 by 100. In some embodiments, storing the correlation matrix as an image includes storing the correlation matrix as a greyscale image for input to a neural network. Applying a machine learning model may include applying an image recognition neural network to the images. The machine learning model may, in some embodiments, be a neural network and the method may include training at least a portion of the neural network based on known genotype to phenotype relationships. In some embodiments, applying a machine learning model includes providing, to the machine learning model, each of multiple k-mer spectral images for a genotype as corresponding channels of a multi-channel input image.

In some embodiments, the machine learning model is a neural network and applying a machine learning model includes modifying the final layer of the neural network to output three values corresponding to probabilities of assignment to a high, medium, and low phenotypic trait value category. In some embodiments, applying a machine learning model includes providing the images to an ensemble of neural networks. In some embodiments, providing the images to an ensemble of neural networks includes providing a subset of the images for a genotype to a first neural network and providing another subset of the images for the genotype to a second neural network. The method may additionally include providing, by the compute device, 3 k-mer images for the genotype to the first neural network and providing a remainder of the k-mer images for the genotype to the second neural network. In some embodiments, determining one or more impactful genomic regions includes generating attribution scores associated with genomic regions. The attribution scores may indicate a degree to which the corresponding region contributed to or detracted from the identification of a relationship between the genotype and the corresponding phenotype.

In some embodiments, determining one or more impactful genomic regions includes utilizing an integrated gradient algorithm to determine attribution scores. Determining one or more impactful genomic regions may include generating attribution images indicative of determined attribution scores. In some embodiments, the method may include identifying, by the compute device, clusters of pixels with values that satisfy a predefined threshold as the impactful regions that underlie the relationship between a genotype and a phenotype. The method may, in some embodiments, include conducting, by the compute device, pathway enrichment analysis based on the impactful genomic regions. Conducting pathway enrichment analysis may include determining whether the impactful genomic regions are statistically associated with known biological pathways. In some embodiments, conducting pathway enrichment analysis includes determining whether the impactful genomic regions represent novel pathways.

In another aspect of the disclosure, one or more machine-readable storage media include a plurality of instructions stored thereon that, in response to being executed, cause a compute device to convert data indicative of genomic variation into images. The instructions may additionally cause the compute device to apply a machine learning model to the images to identify relationships between the genomic variation and phenotypic variation. In addition, the instructions may cause the compute device to determine one or more impactful genomic regions that underlie an identified relationship between a genotype and a phenotype. In some embodiments, the instructions cause the compute device to convert data indicative of genomic variation into images by converting data indicative of genomic variation into greyscale images. In some embodiments, the instructions cause the compute device to translate genome sequence data into k-mers. The instructions may cause the compute device to produce greyscale images indicative of position-indexed k-mers. In some embodiments, the instructions may cause the compute device to convert data indicative of genomic variation into images by generating a defined number of k-mer spectral images for each of multiple strains of an organism. The instructions may be such that generating a defined number of k-mer spectral images for each of multiple strains of an organism includes generating a defined number of k-mer spectral images for each of multiple strains of a plant.

The instructions may cause the compute device to generate a defined number of k-mer spectral images for each of multiple strains of a plant by generating a defined number of k-mer spectral images for each of multiple strains of a crop. In some embodiments, the instructions may cause the compute device to generate a defined number of k-mer spectral images for each of multiple strains of a crop by generating a defined number of k-mer spectral images for each of multiple strains of corn or rice. In some embodiments, the instructions may cause the compute device to utilize long or short read sequence data (e.g. FASTQ-formatted). In other embodiments, the instructions may cause the compute device to utilize one or more variant call format files indicative of variations in a genome from a reference genome. In some embodiments, converting data indicative of genomic variation into images includes utilizing raw genome sequence (FASTA-formatted) data. The instructions may cause the compute device to convert data indicative of genomic variation into images by utilizing k-mer counts of varying size k (e.g., 3, 5, and 7). In some embodiments, the instructions may additionally cause the compute device to split an input file of nucleotide sequences into windows. Further, instructions may cause the compute device to decompose the windows into sub-windows. The instructions may also cause the compute device to concatenate, within each sub-window, reference alleles and variant alleles. Further, the instructions may cause the compute device to calculate k-mers within each sub-window.

The instructions may additionally cause the compute device to compute pairwise Pearson correlation scores among the sub-windows to generate a correlation matrix for each window representing correlations between a variant and a reference sequence. Further, the instructions may cause the compute device to store the correlation matrix as an image for input to the machine learning model. In some embodiments, the instructions cause the compute device to perform pairwise correlations between vectors of k-mer counts to generate a correlation matrix. The length of the vectors and matrix size may be determined by the k-mer size selected and the number of windows and sub-windows specified. For example, in some embodiments, the vectors may be of length 100 and the correlation matrix may be 100 by 100. The instructions may cause the compute device to store the correlation matrix as a greyscale image for input to a neural network. In some embodiments, the instructions may cause the compute device to apply a machine learning model by applying an image recognition neural network to the images. In some embodiments, the machine learning model may be a neural network and the instructions may cause the compute device to train at least a portion of the neural network based on known genotype to phenotype relationships.

The instructions may cause the compute device to provide, to the machine learning model, each of multiple k-mer spectral images for a genotype as corresponding channels of a multi-channel input image. In embodiments in which the machine learning model is a neural network, the instructions may cause the compute device to modify the final layer of the neural network to output three values corresponding to probabilities of assignment to a high, medium, and low phenotypic trait value category. In some embodiments, the instructions may cause the compute device to provide the images to an ensemble of neural networks. The instructions may be such that providing the images to an ensemble of neural networks includes providing a subset of the images for a genotype to a first neural network and providing another subset of the images for the genotype to a second neural network. The instructions may cause the compute device to provide 3 k-mer images for the genotype to the first neural network and provide a remainder of the k-mer images for the genotype to the second neural network. In some embodiments, the instructions may cause the compute device to generate attribution scores associated with genomic regions. The attribution scores may indicate a degree to which the corresponding region contributed to or detracted from the identification of a relationship between the genotype and the corresponding phenotype. The instructions may cause the compute device to utilize an integrated gradient algorithm to determine attribution scores.

In some embodiments, the instructions may cause the compute device to determine one or more impactful genomic regions by generating attribution images indicative of determined attribution scores. The instructions may additionally cause the compute device to identify clusters of pixels with values that satisfy a predefined threshold as the impactful regions that underlie the relationship between a genotype and a phenotype. In some embodiments, the instructions may cause the compute device to conduct pathway enrichment analysis based on the impactful genomic regions. The pathway enrichment analysis may include determining whether the impactful genomic regions are statistically associated with known biological pathways or whether the impactful genomic regions represent novel pathways.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements. The detailed description particularly refers to the accompanying figures in which:

FIG. 1 is a simplified block diagram of at least one embodiment of a system for predicting phenotype and associated biological pathways from genomic variation data;

FIG. 2 is a simplified block diagram of at least one embodiment of a compute device of the system of FIG. 1;

FIGS. 3-6 are simplified block diagrams of at least one embodiment of a method for predicting phenotype and associated biological pathways from genomic variation data that may be executed by the system of FIG. 1;

FIG. 7 is a set of k-mer spectral images for a single genotype that may be produced by the system of FIG. 1;

FIG. 8 is an expanded view of a k-mer spectral image that may be produced by the system of FIG. 1;

FIG. 9 is a diagram of at least one embodiment of a quantitative summary of mean trait values by corresponding assigned trait classes that may be utilized in training one or more machine learning models of the system of FIG. 1;

FIG. 10 is a set of images including a k-mer spectral image and an attribution image indicative of attribution scores that may be produced by the system of FIG. 1;

FIG. 11 is a chart of genome regions that may be determined to be impactful for producing corresponding phenotypes by the system of FIG. 1; and

FIG. 12 is a methodological flowchart for detailing the assignment of biological pathways to regions of a genome that may be utilized in connection with the system of FIG. 1.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

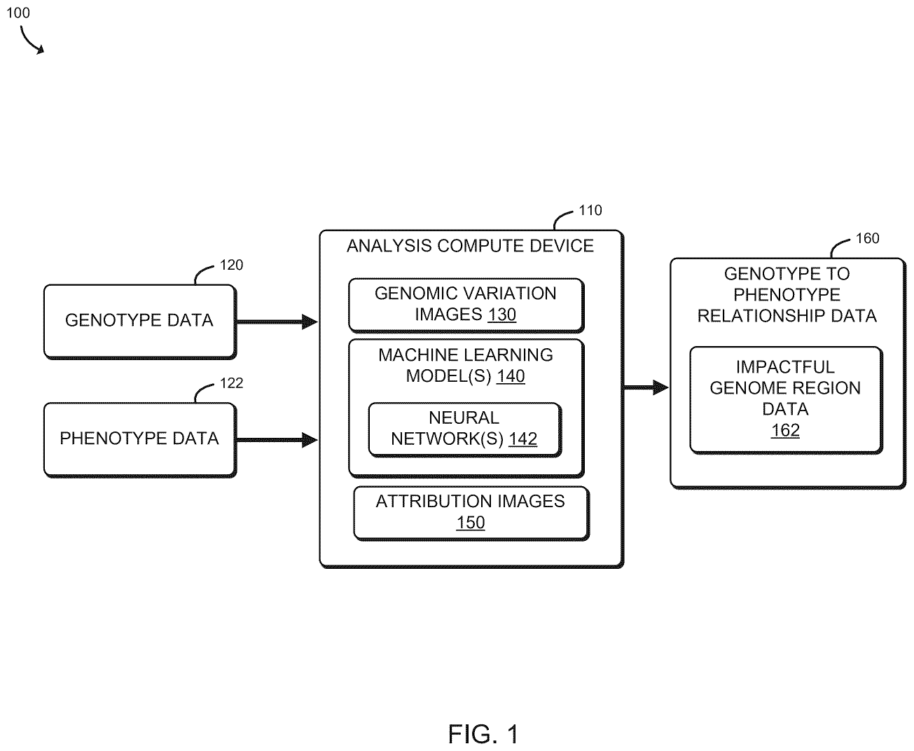

Referring now to FIG. 1, a system 100 for predicting phenotype and associated biological pathways from genomic variation data includes, in the illustrative embodiment, an analysis compute device 110. In the illustrative embodiment, the analysis compute device 110 is configured to obtain (e.g., receive) genotype data 120, which may be embodied as any data indicative of genotypes of an organism. In some embodiments, the genotype data 120 may be embodied as long or short read sequence data, variant call format data, or the like. That is, in some embodiments, the genotype data 120 may not define entire genetic sequences for a corresponding genome and instead may indicate only the variations from a reference genome. Regardless, as a group, the genotype data 120 represents genomic variation among a set of genomes (e.g., strains) of an organism, such as a plant (e.g., a various strains of a crop, such as various strains of rice or corn). As indicated in FIG. 1, the analysis compute device 110 may obtain phenotype data 122, which may be embodied as any data indicative of a set of phenotypes (e.g., traits) associated with the various strains of the organism represented in the genotype data 120.

In operation, the analysis compute device 110 may utilize the genotype data 120 and the phenotype data 122 to train one or more machine learning models 140 based on known relationships between genotypes and phenotypes represented in the genotype data 120 and the phenotype data 122. In at least some embodiments, the machine learning model(s) 140 may include one or more neural networks 142. Once trained to accurately determine whether a given genotype corresponds with a given phenotype, the analysis compute device 110 may produce useful genotype to phenotype relationship data 160, which may be embodied as data indicative of the pathway(s) (e.g., biological causes) between a genotype and a corresponding phenotype. That is, the genotype to phenotype relationship data 160 may include impactful genome region data 162 which may be embodied as data that identifies portions of the genome (e.g., k-mers or sets of k-mers, as described in more detail here) that have been identified (e.g., via a determination of attribution scores and production of corresponding attribution images 150, as described in more detail herein) as significantly contributing to the determination by the machine learning models 140 that a particular genotype will result in a particular phenotype. As such, the genotype to phenotype data may provide the basis for pathway enrichment analysis, by which suspected pathways between genotypes and phenotypes are confirmed and previously unknown (e.g., novel) pathways are discovered.

In performing the operations, the analysis compute device 110 operates on data in the form of images and, in the illustrative embodiment, provides genomic variation images 130 in which k-mers (e.g., nucleotide sequences of length k) and their positional information are represented to the machine learning models 140, which, in the illustrative embodiment, are configured to efficiently perform object recognition, pattern recognition, and computer vision operations, using an accelerator device, such as a graphical processing unit (GPU). As such, unlike typical approaches such as genome wide association studies (GWAS) which typically rely on high performance computing clusters (HPCs), the system 100 enables a computationally more efficient alternative to detecting relationships between genotypes and phenotypes. Additionally, the system 100 can capture potentially large-scale structural variation in DNA (e.g., duplications, deletions, transposition, etc.) that standard GWAS approaches may miss. Further, the system 100, utilizing machine learning (ML), may capture more complex relationships between genes and traits than existing GWAS methods. As such, and considering the volume of existing genetic information to train ML models and the ongoing improvements to ML image processing algorithms, the system 100 represents an improved approach for genotype-to-phenotype modeling over conventional approaches.

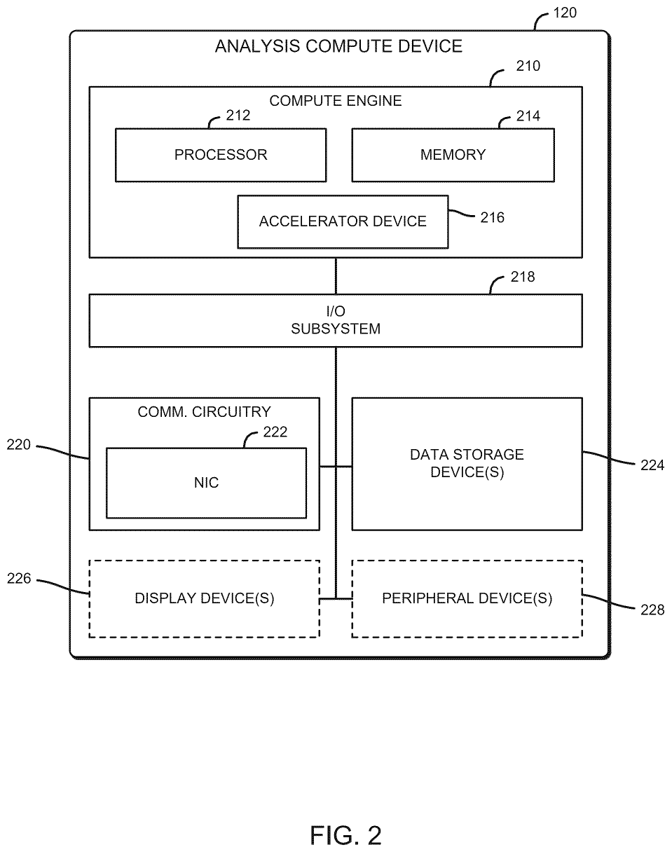

Referring now to FIG. 2, the analysis compute device 110 includes a compute engine 210, an input/output (I/O) subsystem 218, communication circuitry 220, and one or more data storage devices 224. In some embodiments, the analysis compute device 110 may include one or more display devices 226 and/or one or more peripheral devices 228 (e.g., a mouse, a physical keyboard, etc.). In some embodiments, one or more of the illustrative components may be incorporated in, or otherwise form a portion of, another component. The compute engine 210 may be embodied as any type of device or collection of devices capable of performing various compute functions described below. In some embodiments, the compute engine 210 may be embodied as a single device such as an integrated circuit, an embedded system, a field-programmable gate array (FPGA), a system-on-a-chip (SOC), or other integrated system or device. Additionally, in the illustrative embodiment, the compute engine 210 includes or is embodied as a processor 212, a memory 214, and an accelerator device 216 (e.g., circuitry configured to perform a set of operation faster or more efficiently than a general purpose processor 212, such as a graphics processing unit (GPU)). The processor 212 may be embodied as any type of processor capable of performing the functions described herein. For example, the processor 212 may be embodied as a single or multi-core processor(s), a microcontroller, or other processor or processing/controlling circuit. In some embodiments, the processor 212 may be embodied as, include, or be coupled to an FPGA, an application specific integrated circuit (ASIC), reconfigurable hardware or hardware circuitry, or other specialized hardware to facilitate performance of the functions described herein. In some embodiments, the processor 212 may be combined with the accelerator device 216 as an accelerated processing unit (APU).

In embodiments, the processor 212 is capable of receiving, e.g., from the memory 214 or via the I/O subsystem 218, a set of instructions which when executed by the processor 212 cause the analysis compute device 110 to perform one or more operations described herein. In embodiments, the processor 212 is further capable of receiving, e.g., from the memory 214 or via the I/O subsystem 218, one or more signals from external sources, e.g., from the peripheral devices 228 or via the communication circuitry 220 from an external compute device, external source, or external network. As one will appreciate, a signal may contain encoded instructions and/or information. In embodiments, once received, such a signal may first be stored, e.g., in the memory 214 or in the data storage device(s) 224, thereby allowing for a time delay in the receipt by the processor 212 before the processor 212 operates on a received signal. Likewise, the processor 212 may generate one or more output signals, which may be transmitted to an external device, e.g., an external memory or an external compute engine via the communication circuitry 220 or, e.g., to one or more display devices 226. In some embodiments, a signal may be subjected to a time shift in order to delay the signal. For example, a signal may be stored on one or more storage devices 224 to allow for a time shift prior to transmitting the signal to an external device. One will appreciate that the form of a particular signal will be determined by the particular encoding a signal is subject to at any point in its transmission (e.g., a signal stored will have a different encoding than a signal in transit, or, e.g., an analog signal will differ in form from a digital version of the signal prior to an analog-to-digital (A/D) conversion).

The main memory 214 may be embodied as any type of volatile (e.g., dynamic random access memory (DRAM), etc.) or non-volatile memory or data storage capable of performing the functions described herein. Volatile memory may be a storage medium that requires power to maintain the state of data stored by the medium. In some embodiments, all or a portion of the main memory 214 may be integrated into the processor 212. In operation, the main memory 214 may store various software and data used during operation such as genotype data, phenotype data, genomic variation images, one or more machine learning models, attribution images, genotype to phenotype relationships data, applications, libraries, and/or drivers.

The compute engine 210 is communicatively coupled to other components of the analysis compute device 110 via the I/O subsystem 218, which may be embodied as circuitry and/or components to facilitate input/output operations with the compute engine 210 (e.g., with the processor 212, the main memory 214, and the accelerator device 216) and other components of the analysis compute device 110. For example, the I/O subsystem 218 may be embodied as, or otherwise include, memory controller hubs, input/output control hubs, integrated sensor hubs, firmware devices, communication links (e.g., point-to-point links, bus links, wires, cables, light guides, printed circuit board traces, etc.), and/or other components and subsystems to facilitate the input/output operations. In some embodiments, the I/O subsystem 218 may form a portion of a system-on-a-chip (SoC) and be incorporated, along with one or more of the processor 212, the main memory 214, the accelerator device 216 and other components of the analysis compute device 110, into the compute engine 210.

The communication circuitry 220 may be embodied as any communication circuit, device, or collection thereof, capable of enabling communications over a network between the analysis compute device 110 and another device. The communication circuitry 220 may be configured to use any one or more communication technology (e.g., wired or wireless communications) and associated protocols (e.g., Ethernet, Wi-Fi®, WiMAX, Bluetooth®, etc.) to effect such communication.

The illustrative communication circuitry 220 includes a network interface controller (NIC) 222. The NIC 222 may be embodied as one or more add-in-boards, daughter cards, network interface cards, controller chips, chipsets, or other devices that may be used by the analysis compute device 110 to connect with another compute device. In some embodiments, the NIC 222 may be embodied as part of a system-on-a-chip (SoC) that includes one or more processors, or included on a multichip package that also contains one or more processors. In some embodiments, the NIC 222 may include a local processor (not shown) and/or a local memory (not shown) that are both local to the NIC 222. Additionally or alternatively, in such embodiments, the local memory of the NIC 222 may be integrated into one or more components of the analysis compute device 110 at the board level, socket level, chip level, and/or other levels.

Each data storage device 224, may be embodied as any type of device configured for short-term or long-term storage of data such as, for example, memory devices and circuits, memory cards, hard disk drives, solid-state drives, or other data storage device. Each data storage device 224 may include a system partition that stores data and firmware code for the data storage device 224 and one or more operating system partitions that store data files and executables for operating systems.

Each display device 226 may be embodied as any device or circuitry (e.g., a liquid crystal display (LCD), a light emitting diode (LED) display, a cathode ray tube (CRT) display, etc.) configured to display visual information (e.g., text, graphics, etc.) to a user. In some embodiments, a display device 226 may be embodied as a touch screen (e.g., a screen incorporating resistive touchscreen sensors, capacitive touchscreen sensors, surface acoustic wave (SAW) touchscreen sensors, infrared touchscreen sensors, optical imaging touchscreen sensors, acoustic touchscreen sensors, and/or other type of touchscreen sensors) to detect selections of on-screen user interface elements or gestures from a user.

In the illustrative embodiment, the components of the analysis compute device 110 are housed in a single unit. However, in other embodiments, the components may be in separate housings, in separate racks of a data center, and/or spread across multiple data centers or other facilities. Further, it should be appreciated that the analysis compute devices 110 may include other components, sub-components, and devices commonly found in a computing device, which are not discussed above in reference to the analysis compute device 110 and not discussed herein for clarity of the description.

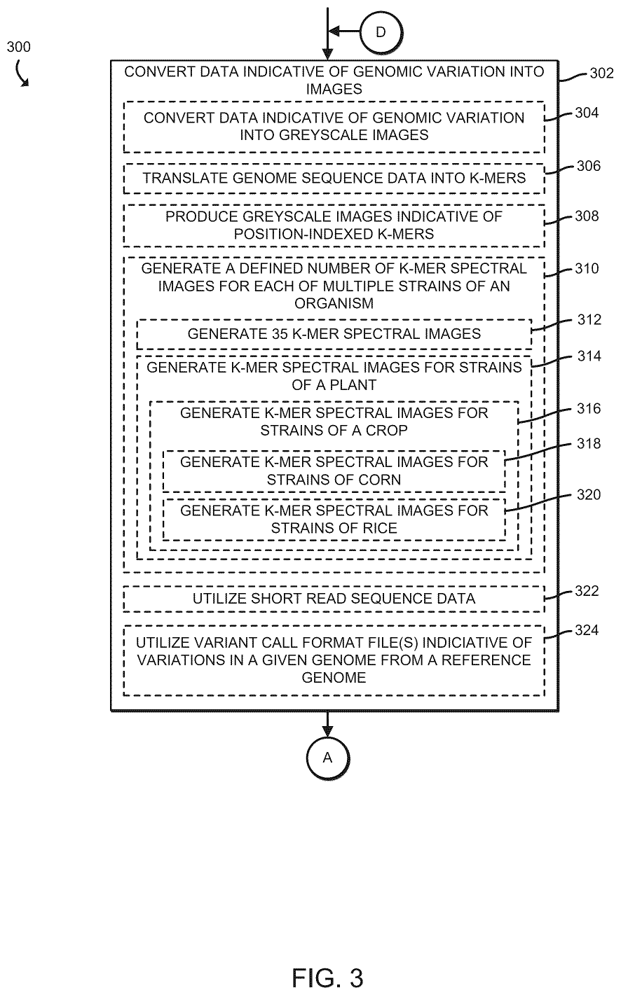

Referring now to FIG. 3, the system 100, and specifically, the analysis compute device 110, in the illustrative embodiment, may perform a method 300 for predicting phenotype and associated biological pathways from genomic variation data. The method 300 begins with block 302 in which the analysis compute device 110 converts data indicative of genomic variation into images. In doing so, and as indicated in block 304, the analysis compute device 110 may convert data indicative of genomic variation into grayscale images. In the illustrative embodiment, the analysis compute device 110 translates genome sequence data into k-mers (e.g., nucleotide sequences of length k), as indicated in block 306. In producing the images, the analysis compute device 110 may produce greyscale images that are indicative of position-indexed k-mers (e.g., the images indicate the positions of the k-mers within the corresponding genome sequence, such as by the positions of the corresponding pixels (representing the k-mers) within the images), as indicated in block 308.

In the illustrative embodiment, the analysis compute device 110 generates a defined number of k-mer spectral images for each of multiple strains of an organism, as indicated in block 310. For example, in some embodiments, the analysis compute device 110 may generate 35 k-mer spectral images for a given genotype (e.g., corresponding to a strain of the organism), as indicated in block 312. The number of k-mer spectral images may vary in different embodiments. As indicated in block 314, the analysis compute device 110 may generate k-mer spectral images for strains of a plant (e.g., the organism is a plant). More specifically, in some embodiments, the analysis compute device 110 may generate k-mer spectral images for strains of a crop, as indicated in block 316. For example, the analysis compute device 110 may generate k-mer spectral images for strains of corn, as indicated in block 318. In other embodiments, the analysis compute device 110 may generate k-mer spectral images for strains of rice, as indicated in block 320. In some embodiments, the analysis compute device 110 may utilize (e.g., as the input genotype data 120) short read sequence data (e.g., data sets in which a genome has been sectioned into sets of 50 to 300 bases), as indicated in block 322. In other embodiments, the analysis compute device 110 may utilize long read sequence data. In some embodiments, the analysis compute device 110 may utilize one or more variant call format (VCF) files as the input genotype data (genomic variation data) 120. Variant call format files may be embodied as data sets that indicate the differences (e.g., variations) of a given genome from a reference genome (e.g., rather than reproducing the entire genome). In some embodiments, a VCF file can be in table format containing about 17 million lines. Each line of a VCF file can represent a single variant and include, among other data, a reference sequence and a variant sequence.

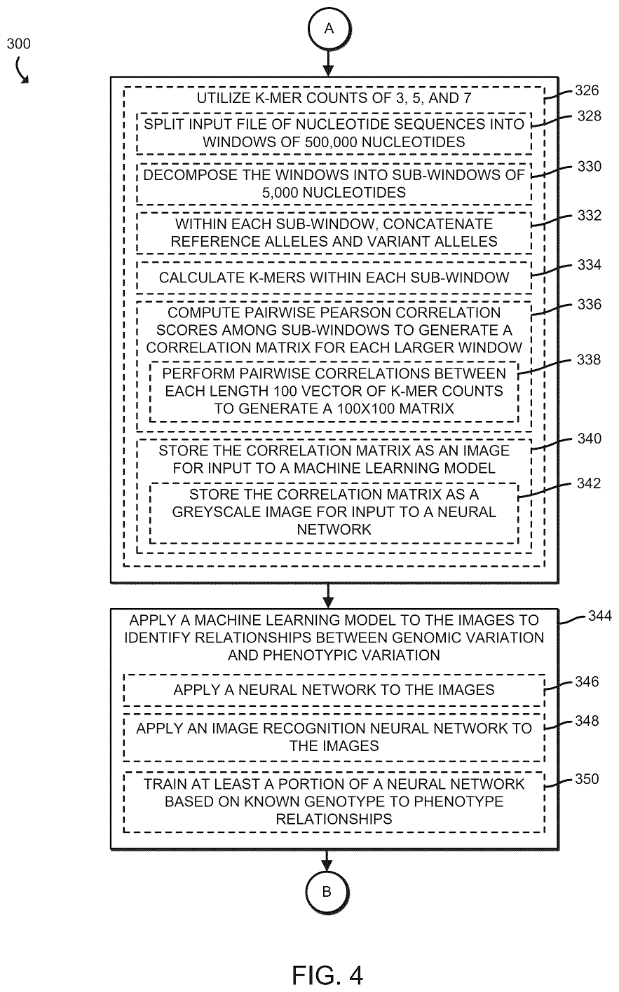

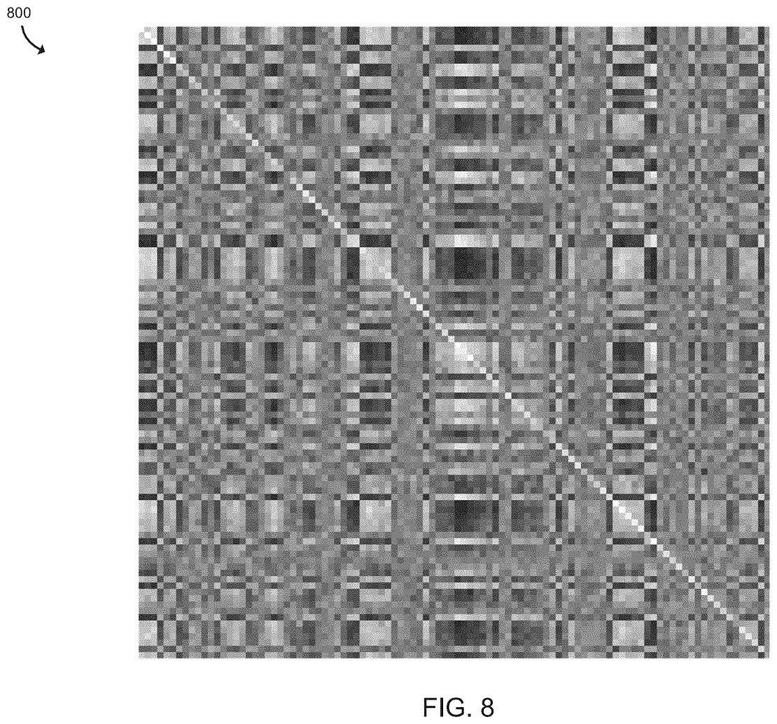

Referring now to FIG. 4, continuing the method 300, in some embodiments, the analysis compute device 110 may utilize k-mer counts of 3, 5, and 7 (e.g., nucleotide sequences of length 3, 5, and 7), as indicated in block 326. The k-mer counts may vary across embodiments. The analysis compute device 110 may split an input file (e.g., genotype data 120) of nucleotide sequences into windows (e.g., of 500,000 nucleotides), as indicated in block 328. The analysis compute device 110 may further decompose those windows (e.g., from block 328) into sub-windows (e.g., of 5,000 nucleotides each), as indicated in block 330. The numbers of nucleotides in the windows and sub-windows may vary depending on the embodiment. For example, in some embodiments, the analysis compute device 110 may split a VCF file into windows of 500,000 lines each and further into sub-windows of 5,000 lines each. Each line of the VCF file can include a reference allele and a variant allele and each allele can include one or more nucleotides. Additionally, within each sub-window, the analysis compute device 110 may concatenate reference alleles and variant alleles, as indicated in block 332. The analysis compute device 110 may calculate the k-mers within each sub-window (e.g., from block 330), as indicated in block 334. Further, in generating the images, the analysis compute device 110 may compute pairwise Pearson correlation scores among the sub-windows to generate a correlation matrix for each larger window (e.g., from block 328), as indicated in block 336. In computing the Pearson correlation scores, the analysis compute device 110 may perform pairwise correlations between each length-100 vector of k-mer counts to generate a 100×100 matrix, as indicated in block 338. In other embodiments, the vector length and matrix dimensions may vary. Further, the analysis compute device 110 may store the resulting correlation matrix (e.g., for each window) as an image for input to a machine learning model (e.g., the machine learning model(s) 140 of FIG. 1), as indicated in block 340. In doing so, the analysis compute device 110 may store the resulting correlation matrix as a greyscale image for input to a neural network (e.g., a neural network 142 of FIG. 1), as indicated in block 342. The images described above, in the illustrative embodiment, are the genomic variation images 130 described above with respect to FIG. 1. An embodiment of a set of k-mer spectral images 700 (e.g., genomic variation images 130) that may be produced by the analysis compute device 110 according to the operations described above for a single genotype is shown. In the illustrative embodiment, the analysis compute device 110 produces corresponding sets of k-mer spectral images (e.g., genomic variation images 130) for each of multiple genotypes. FIG. 8 provides an extended (e.g., enlarged) view of a k-mer spectral image 800 that may be produced by the analysis compute device 110.

In the illustrative embodiment, the method 300 continues to block 344, in which the analysis compute device 110 applies a machine learning model (e.g., a machine learning model 140) to the images (e.g., the genomic variation images 130) to identify relationships between genomic variation and phenotypic variation. In doing so, and as indicated in block 346, the analysis compute device 110 may apply a neural network (e.g., a neural network 142) to the images, as indicated in block 346. As indicated in block 348, the analysis compute device 110 may apply an image recognition neural network to the images (e.g., the neural network 142 may be a neural network (e.g., EfficientNet-B7 and/or EfficientNet-B0 neural networks) trained to recognize images). In some embodiments, the neural network comprises multiple networks that process different channels in the images. For example, a first 3 channels of a 36-channel image can be fed through one network and the remaining 33 channels can be fed through another network. The machine learning model 140 (e.g., neural network 142) may be pre-trained to recognize images, however, the analysis compute device 110 may train at least a portion of the machine learning model 140 (e.g., neural network 142) based on known genotype to phenotype relationships (e.g., to accurately predict a relationship between a genotype and a phenotype), as indicated in block 350.

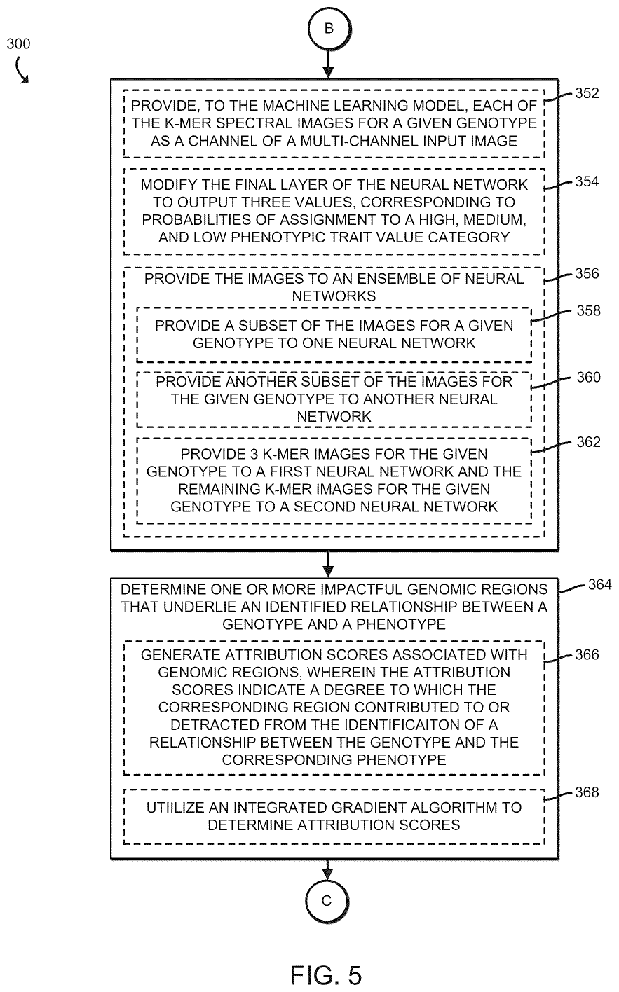

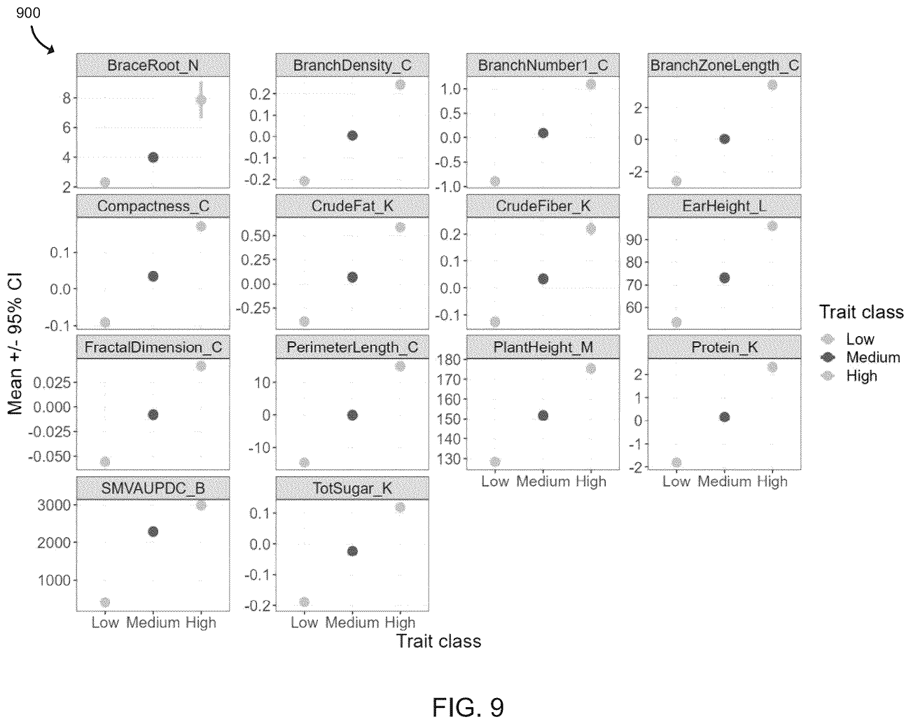

Referring now to FIG. 5, the analysis compute device 110 may provide, to the machine learning model 140 (e.g., the neural network 142), each of the k-mer spectral images (e.g., the genomic variation images 130) for a given genotype (e.g., strain of an organism) as a channel of a multi-channel input image, as indicated in block 352. That is, the analysis compute device 110 may treat the 35 greyscale k-mer spectral images associated with a genotype as a single image with 35 channels for by the image recognition machine learning model(s) 140. As indicated in block 354, the analysis compute device 110 may modify the final layer of the neural network to output three values (rather than a default number of values, such as 1,000 values). Those values, in the illustrative embodiment, correspond to probabilities of assignment to a high, medium, and low phenotypic trait value category. That is, in some embodiments, the trained model analyzes an input genomic image and then assigns three probability scores for the different trait levels. For example, the probability scores may indicate a 10% chance that a given strain is “low ear height”, a 60% chance that the strain is “medium ear height”, and a 30% chance that the strain is “high ear height”. In other embodiments, the number of output values and what they represent may vary. FIG. 9 provides a quantitative summary 900 of mean trait values by corresponding assigned trait classes that may be utilized in training one or more machine learning models 140 of the analysis compute device 110. That is, in an embodiment in which the analysis compute device 110 is configured to identify relationships between genotypes and phenotypes (e.g., traits) of corn, the set of known phenotypes may be down selected (e.g., from 162 phenotypes to 15 phenotypes), and for each of the phenotypes, the genotypes may be assigned a label of “high”, “medium”, or “low” based on a cluster analysis among genotype values. For example, genotypes with greater crude fat content than a typical genotype, for instance, may be assigned a value of “high”, those with an intermediate value may be assigned a value of “medium”, and those with a lower crude value than a typical genotype may be assigned a value of “low”. The summary 900 shows strong separation of genotypes along trait axes, and as such, the trait labels capture real phenotypic variation across the entire distribution.

In some embodiments, the analysis compute device 110 may provide the images (e.g., the genomic variation images 130) to an ensemble (e.g., combination) of neural networks 142, as indicated in block 356. In doing so, and as indicated in block 358, the analysis compute device 110 may provide a subset of the images for a given genotype to one neural network, and may provide another subset of the images for that same genotype to another neural network (e.g., concurrently), as indicated in block 360. As indicated in block 362, the analysis compute device 110 may provide, for example, 3 k-mer images for a given genotype to one neural network (e.g., as three channels) and the remaining k-mer images (e.g., remaining 32 images) to another neural network (e.g., as 32 channels). The analysis compute device 110 may combine the outputs of the multiple neural networks as a final output (e.g., indicative of the determined level of correlation between the genotype and a phenotype).

In the illustrative embodiment, the method 300 continues in block 364, in which the analysis compute device 110 determines one or more impactful genomic regions that underlie an identified relationship between a genotype and phenotype. In doing so, the analysis compute device 110 may generate attribution scores associated with genomic regions, as indicated in block 366. The attribution scores, in the illustrative embodiment, indicate a degree to which the corresponding region (e.g., set of k-mers) contributed to or detracted from the identification (e.g., by the machine learning model(s) 140) of a relationship between the genotype and the corresponding phenotype (e.g., determined to have a high level of correlation). The analysis compute device 110 may utilize an integrated gradient algorithm to determine the attribution scores, as indicated in block 368. In other embodiments, the analysis compute device 110 may utilize a different algorithm to produce the attribution scores.

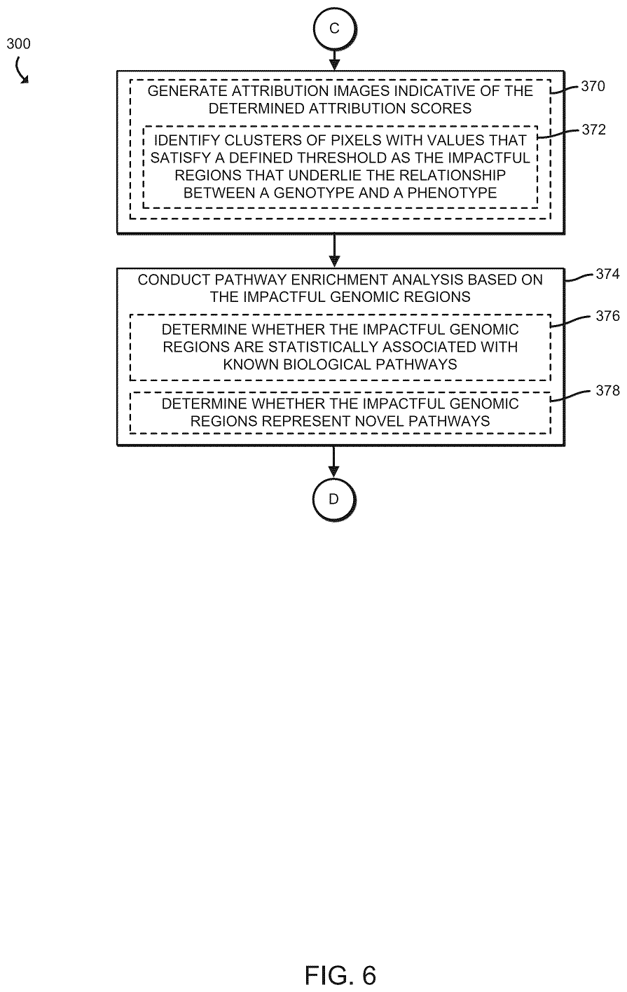

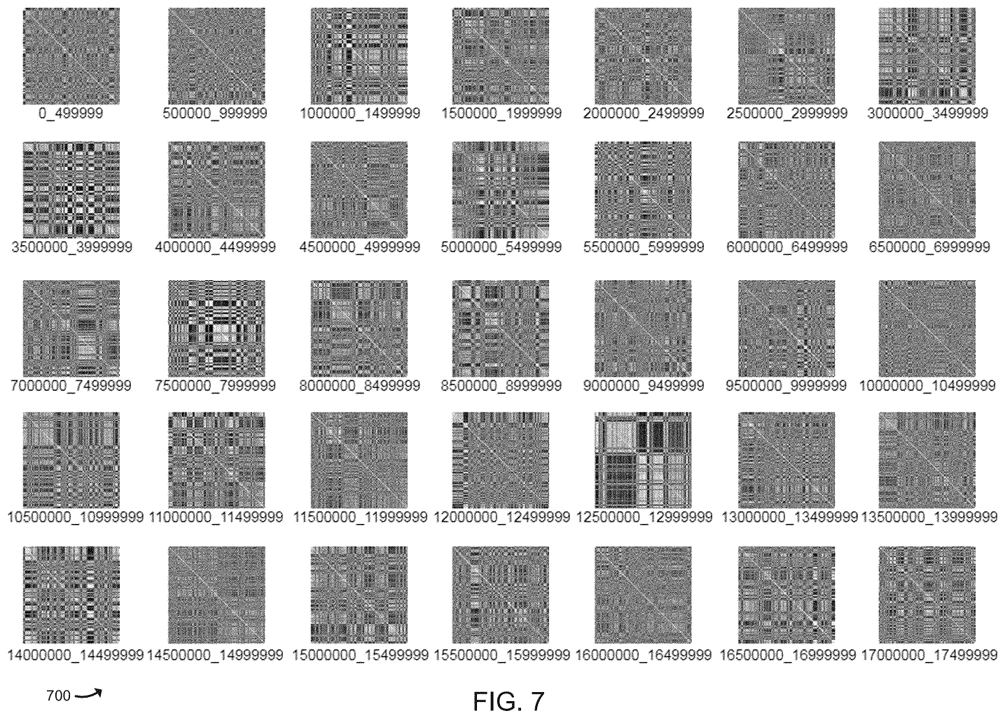

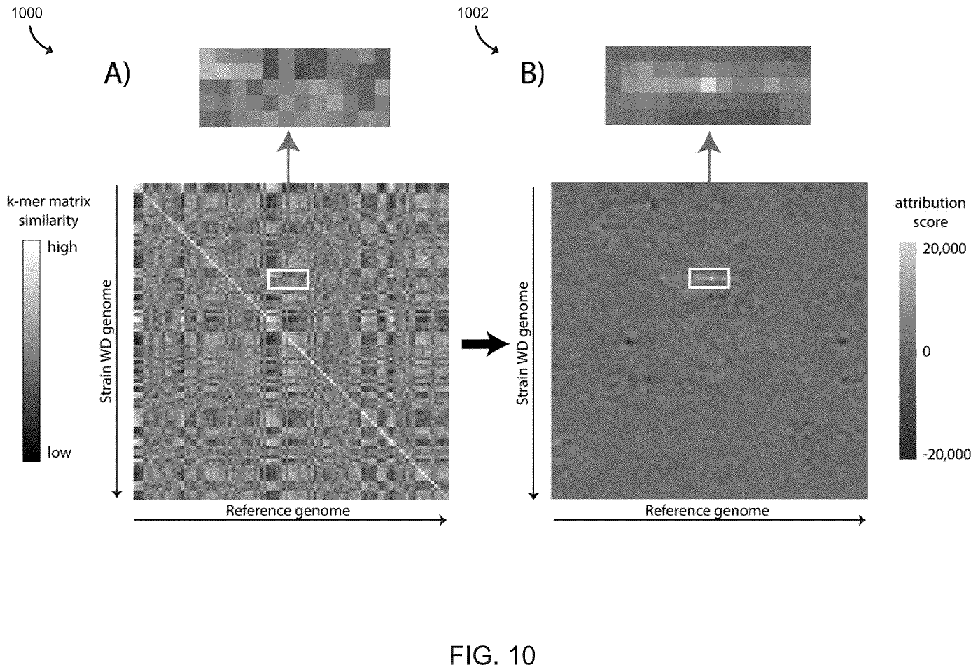

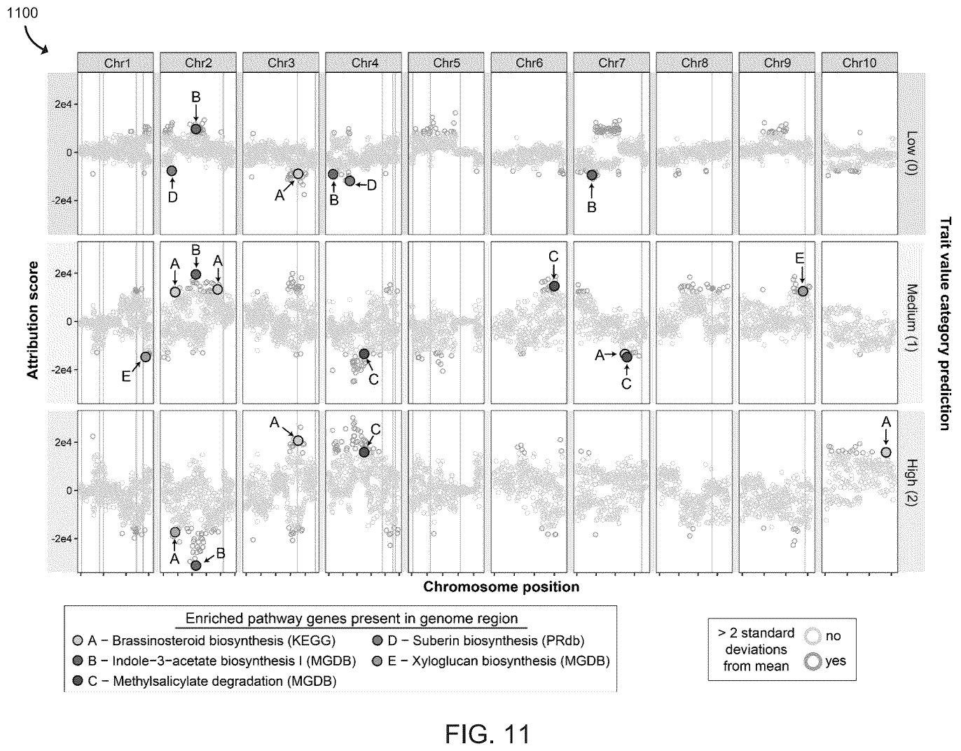

Referring now to FIG. 6, the analysis compute device 110, in the illustrative embodiment, generates attribution images (e.g., heat plots) indicative of the determined attribution scores, as indicated in block 370. An attribution image, in the illustrative embodiment, has, for each pixel, a value (e.g., an intensity) indicative of the corresponding attribution score for a given k-mer or set of k-mers associated with that location (e.g., wherein the location is based on the location(s) of the k-mers represented in the genomic variation images 130 (e.g., the k-mer spectral images)). FIG. 10 illustrates a k-mer spectral image 1000 and an attribution image 1002 indicative of attribution scores that may be produced by the analysis compute device 110 in accordance with the operations described herein. As indicated in block 372, the analysis compute device 110 may identify clusters of pixels with values that satisfy a defined threshold (e.g., the top 5%) as the impactful regions that underlie the relationship between a genotype and a phenotype (e.g., that most contributed to the determination that the genotype corresponds to a particular phenotype). In some embodiments, the analysis compute device 110 may also identify clusters of pixels (e.g., the bottom 5%) as regions that least contributed to (e.g., detracted from) the determination that the genotype corresponds to a particular phenotype. Those regions (e.g., clusters) in the attribution images may be indicative of pathways between the genotype and the phenotypes. For example, a given set of k-mers represented in an attribution image as being particularly impactful may cause a specific metabolite to be synthesized that results in the corresponding phenotype (e.g., trait). FIG. 11 illustrates a chart 1100 of impactful genome regions that may be identified with the analysis compute device 110, in an embodiment. Regional scores for each of three categorical trait value predictions are presented separately in the top, middle, and bottom panels as indicated. Each vertical panel represents 1 of 10 main Z. mays chromosomes as named at the top. The 17 vertical lines indicate regions identified as informative in their GWAS study of the same genotypes and trait data. Regions flagged as potentially informative are identified according to the bottom right legend. Informative regions containing genes contained in statistically enriched pathways are flagged with circles and labeled according to the legend at the bottom left.

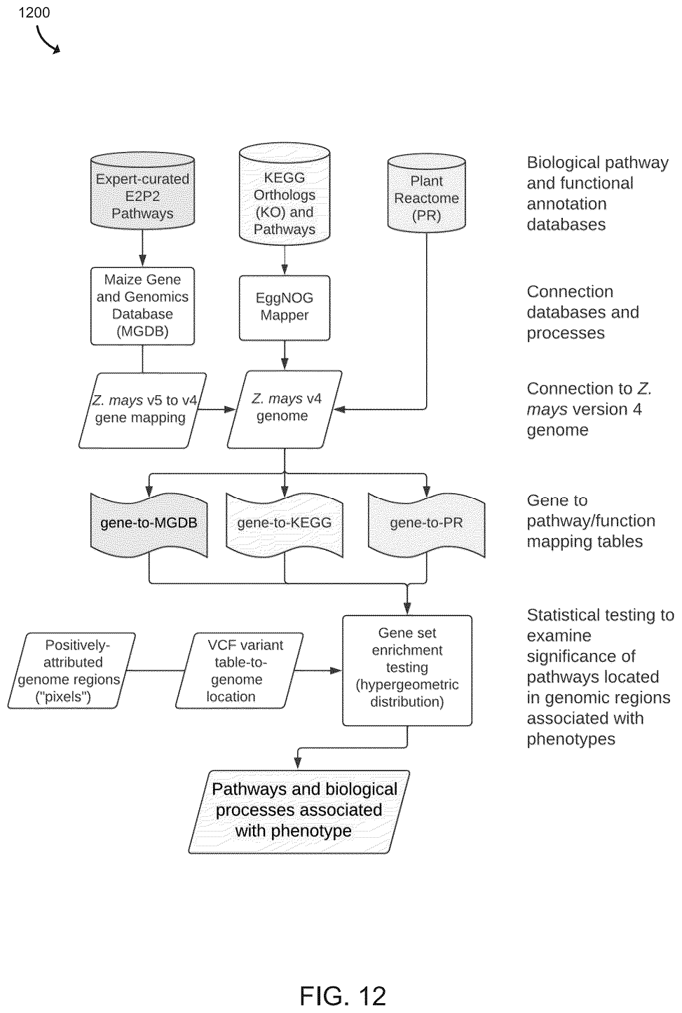

The method 300 may continue in block 374, in which the analysis compute device 110 conducts or is used to conduct pathway enrichment analysis based on the impactful genomic regions (e.g., from block 364). In doing so, the analysis compute device 110 may determine or be used to determine whether the impactful genomic regions are statistically associated with known biological pathways (e.g., to confirm the accuracy of the determinations made by the machine learning model(s) 140 or to confirm a suspected pathway), as indicated in block 376. Additionally or alternatively, the analysis compute device 110 may determine or be used to determine whether one or more impactful genomic regions represents a novel pathway (e.g., a previously unknown pathway), as indicated in block 378. FIG. 12 provides a methodological flowchart 1200 for an embodiment of a method that may be used in connection with the analysis compute device 110 for assigning biological pathways to regions of a genome. The flowchart 1200 is illustrative of a process for assigning pathways for corn. In the embodiment, annotation of corn pathways may be supplemented by the Plant Reactome database (PRdb), the Kyoto Encyclopedia of Genes and Genomes (KEGG), and/or the Maize Genetics and Genomics Database (MGDB) generated by using an Ensemble Enzyme Prediction Pipeline (E2P2) of the strain B73 Z. mays genome. The gene to pathways assignment is based on version 5 of the corn genome but illustrates a process for transference of annotations from version 5 to version 4 of the corn genome, which is used in a portion of the operations. In some embodiments, for the selection of “meaningful” (e.g., impactful) genome region attribution scores (those considered to contribute substantially to the neural network predictions), minimum (negative) and maximum (positive) attribution scores more than two standard deviations above or below the mean value may be designated as “meaningful” in regard to biological pathway determinations. In these corresponding genome region subsets, the genes and associated biological pathways may be extracted. Pathway enrichments may be performed via hypergeometric testing between the subset of pathway annotations encoded in informative genome regions and in the genome as a whole.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

1. A method comprising:
converting, by a compute device, data indicative of genomic variation into images;
applying, by the compute device, a machine learning model to the images to identify relationships between the genomic variation and phenotypic variation; and
determining, by the compute device, one or more impactful genomic regions that underlie an identified relationship between a genotype and a phenotype.

2. The method of claim 1, wherein converting data indicative of genomic variation into images comprises converting data indicative of genomic variation into greyscale images.

3. The method of claim 1, wherein converting data indicative of genomic variation into images comprises translating genome sequence data into k-mers.

4. The method of claim 1, wherein converting data indicative of genomic variation into images comprises producing greyscale images indicative of position-indexed k-mers.

5. The method of claim 1, wherein converting data indicative of genomic variation into images comprises generating a defined number of k-mer spectral images for each of multiple strains of an organism.

6. The method of claim 5, wherein generating the defined number of k-mer spectral images for each of multiple strains of the organism comprises generating a defined number of k-mer spectral images for each of multiple strains of a plant.

7. The method of claim 6, wherein generating the defined number of k-mer spectral images for each of multiple strains of the plant comprises generating a defined number of k-mer spectral images for each of multiple strains of a crop.

8. The method of claim 7, wherein generating the defined number of k-mer spectral images for each of multiple strains of the crop comprises generating a defined number of k-mer spectral images for each of multiple strains of corn or rice.

9. The method of claim 1, wherein converting data indicative of genomic variation into images comprises utilizing long or short read sequence data.

10. The method of claim 1, wherein converting data indicative of genomic variation into images comprises utilizing one or more variant call format files indicative of variations in a genome from a reference genome.

11. The method of claim 1, wherein converting data indicative of genomic variation into images comprises utilizing k-mer counts of 3, 5, and 7.

12. The method of claim 11, further comprising:
splitting, by the compute device, an input file of nucleotide sequences into windows;
decomposing, by the compute device, the windows into sub-windows;
concatenating, by the compute device and within each sub-window, reference alleles and variant alleles; and
calculating, by the compute device, k-mers within each sub-window.

13. The method of claim 12, further comprising:
computing, by the compute device, pairwise Pearson correlation scores among the sub-windows to generate a correlation matrix for each window; and
storing, by the compute device, the correlation matrix as an image for input to the machine learning model.

14. The method of claim 13, wherein computing pairwise Pearson correlation scores comprises performing pairwise correlations between each of multiple length 100 vectors of k-mer counts to generate a 100 by 100 matrix.

15. The method of claim 14, wherein storing the correlation matrix as an image comprises storing the correlation matrix as a greyscale image for input to a neural network.

16. The method of claim 1, wherein applying the machine learning model comprises applying an image recognition neural network to the images.

17. The method of claim 1, wherein the machine learning model is a neural network and the method further comprises training at least a portion of the neural network based on known genotype to phenotype relationships.

18. The method of claim 1, wherein applying the machine learning model comprises providing, to the machine learning model, each of multiple k-mer spectral images for a genotype as corresponding channels of a multi-channel input image.

19. The method of claim 1, wherein the machine learning model is a neural network and wherein applying a machine learning model comprises modifying a final layer of the neural network to output three values corresponding to probabilities of assignment to a high, medium, and low phenotypic trait value category.

20. The method of claim 1, wherein applying the machine learning model comprises providing the images to an ensemble of neural networks.

21. The method of claim 20, wherein providing the images to the ensemble of neural networks comprises providing a subset of the images for a genotype to a first neural network and providing another subset of the images for the genotype to a second neural network.

22. The method of claim 21, further comprising providing, by the compute device, 3 k-mer images for the genotype to the first neural network and providing a remainder of the k-mer images for the genotype to the second neural network.

23. The method of claim 1, wherein determining the one or more impactful genomic regions comprises generating attribution scores associated with genomic regions, wherein each attribution score indicates a degree to which an associated genomic region contributed to or detracted from the identified relationship between the genotype and the phenotype.

24. The method of claim 1, wherein determining the one or more impactful genomic regions comprises utilizing an integrated gradient algorithm to determine attribution scores.

25. The method of claim 1, wherein determining the one or more impactful genomic regions comprises generating attribution images indicative of determined attribution scores.

26. The method of claim 25, further comprising identifying, by the compute device, clusters of pixels in the attribution images with values that satisfy a predefined threshold as the one or more impactful regions that underlie the identified relationship between the genotype and the phenotype.

27. The method of claim 1, further comprising conducting, by the compute device, pathway enrichment analysis based on the one or more impactful genomic regions.

28. The method of claim 27, wherein conducting pathway enrichment analysis comprises determining whether the one or more impactful genomic regions are statistically associated with known biological pathways.

29. The method of claim 27, wherein conducting pathway enrichment analysis comprises determining whether the one or more impactful genomic regions represent novel pathways.
